(19) United States
(12) Patent Application Publication
(10) Pub. No.: US 2006/0263818 A1
(43) Pub. Date: Nov. 23, 2006

(54) High throughput multi-antigen microfluidic fluorescence immunoassays

(76) Inventors: Axel Scherer, Laguna Beach, CA (US); Emil P. Kartalov, Los Angeles, CA (US); W. French Anderson, San Gabriel, CA (US); Clive Taylor, South Pasadena, CA (US)

Correspondence Address: Daniel L. Dawes;Myers Dawes Andras & Sherman LLP, 19900 MacArthur Boulevard, Irvine, CA, 92612, US

(21) Appl. No.: 11/439,288

(22) Filed: May 22, 2006

Related U.S. Application Data
(60) Provisional application No. 60/683,822, filed on May 23, 2005.

(51) Int. Cl. C12Q 1/68; G01N 33/53; C12M 1/34

(52) U.S. Cl. 435/6; 435/7.1; 435/287.2

(57) ABSTRACT

The development of a high-throughput multi-antigen microfluidic fluorescence immunoassay system is illustrated in a 100-chamber PDMS (polydimethylsiloxane) chip which performs up to 5 tests for each of 10 samples. Specificity of detection is demonstrated and calibration curves produced for C-Reactive Protein (CRP), Prostate Specific Antigen (PSA), ferritin, and Vascular Endothelial Growth Factor (VEGF). The measurements show sensitivity at and below levels that are significant in current clinical laboratory practice (with SIN>8 at as low as 10 pM antigen concentration). The chip uses 100 nL per sample for all four tests and provides an improved instrument for use in scientific research and “point-of-care” testing in medicine.

### RELATED APPLICATIONS

The present application is related to U.S. Provisional Patent Application Ser. No. 60/683,822, filed on May 23, 2005, which is incorporated herein by reference and to which priority is claimed pursuant to 35 USC 119.

### GOVERNMENT RIGHTS

Financial support was provided for the invention by the National Institutes of Health under NIH Grant no. 1R01 HG002644. The U.S. Government has certain rights to the invention.

### BACKGROUND OF THE INVENTION

1. Field of the Invention

The invention relates to the field of microfluidic circuits and methods used to perform immunoassays.

2. Description of the Prior Art

The ongoing revolution in biological sciences has generated high hopes for the advent of true personalized/preventive medicine. While the necessary biological tools are being developed at a fast pace, it has become clear that their cost, operation, and manufacturability are equally challenging issues that must be solved before the new methods can be widely accepted in medical practice. In the particular case of diagnostics, decentralized “near-patient” or “point-of-care” testing (1) has attempted to provide fast quantitative results at the bedside or in the clinic, thereby decreasing hospital stays, eliminating transportation and administrative expenses, and decreasing errors from mishandling and miscommunication. While a few single-analyte systems (1) have been developed (e.g., the now commonplace Glucometer®), the enormous potential for decentralized testing remains untapped because the vast majority of medical diagnostics is still conducted in clinical laboratories and with the use of large equipment.

A way for ubiquitous near-patient and point-of-care testing to reach fruition is for the current biological techniques to be reduced from the macroscale to the microscale, in a multi-analyte high-throughput format, preferably on handheld devices. In particular, reducing immunoassays to microfluidic scales has been extensively explored in recent years. Many approaches have been proposed, involving glass, TiO₂, silicon, and silicone devices, but none possesses all of the desirable qualities: (i) capability to measure multiple antigens and samples per device, (ii) industrially feasible fabrication, (iii) parsimony of sample and reagents, (iv) adequate sensitivity and specificity, and (v) good reliability and reproducibility.

High kit and instrumentation costs dictate centralization of measurements in large clinical or reference laboratories, resulting in transportation and batch delays of up to 14 days between the phlebotomist appointment and the final availability of the test results. Such delays and the macroscale of samples and reagents drive up the expenses in today's fast-paced and expensive healthcare environment.

### BRIEF SUMMARY OF THE INVENTION

The illustrated embodiment of the invention is a high-throughput multi-antigen microfluidic fluorescence immunoassay system. A 100-chamber polydimethylsiloxane (PDMS) chip performs up to 5 tests for each of 10 samples. In this particular study system, the specificity of detection was demonstrated, and calibration curves were produced for C-reactive protein (CRP), prostate-specific antigen (PSA), ferritin, and vascular endothelial growth factor (VEGF). The measurements show sensitivity at and below clinically normal levels (with a signal-to-noise ratio >8 at as low as 10 pM antigen concentration). The chip uses 100 nL per sample for all tests. The developed system is an important step toward derivative immunoassay applications in scientific research and “point-of-care” testing in medicine.

The circuits or chips of the illustrated embodiment multiplex an immunoassay scheme to allow five simultaneous tests for each of ten samples. Micromechanical valves direct the pressure-driven reagent flow as desired along a network of 10 μm-tall flow channels. “Four-way” valving at each intersection in the test matrix forms a capture microchamber at the intersection of crossing flow channels within which capture microchamber the immunoassay stack is built for a particular sample-test combination.

The basic steps of the assay are as follows. First, monoclonal antibodies flowing in horizontal flow channels from antigen inputs to derivitization exhausts respectively bind to the epoxide coating of the floor of microchamber. Appropriate ones of the valves are opened and closed by providing pressure to a selected one of control ports to isolate the antibody flow from unused portions of the flow channels of chip. It should be noted that the manner of actuation of valves is non-exclusive of other possibilities, e.g. electric actuation; thus, the approach is not limited to pneumatically controlled valves.

Second, by providing pressure to a selected one of control ports to again isolate flow from unused portions of the flow channels of chip, a Tris buffer flowing in horizontal flow channels is used to flushed from a buffer input to derivitization exhausts and from a sample input flowing in vertical flow channels to sample exhausts to inactivate remaining epoxide groups which have not bound to an antigen. Other surface chemistry bindings are also possible, e.g. carboxylate surface binding amino groups in the presence of catalyst, and thus the approach is not limited to epoxide chemistry. In fact, the same is in principle doable on PDMS surfaces, in view of ref. Kartalov et al., Nucleic Acids Research (2004).

Third, samples are fed in vertical flow channels in parallel from sample inputs to sample exhausts. Again pressure is provided to a selected one of control ports to again isolate flow from the portions of the flow channels of chip not used for this purpose. But valve actuation could also be accomplished by other means to the same result.

Fourth, the sample which is then in place in the central portion of matrix is pumped along closed paths or coliseums through the capture microchambers. The coliseum communicates with two microchambers and has a total volume of 10 nL which allows the captured sample to be volumetrically quantized, which is advantageous, if not essential, to making a practical quantified measurement of the sample analyte. It is to be understood that the arrangement of coliseum is shown by way of example only and that other configurations can be employed without departing from the scope and spirit of the invention. For example, a single or more than two microchambers may be communicated to the coliseum, the coliseum may be provided with a different pumping arrangement or volume, and/or an oscillating flow pattern might be employed instead of a circulating pattern.

Fifth, biotinylated polyclonal antibodies fed from antibody inputs flow along horizontal flow channels to derivitization exhausts to complete the immunostacks in the microchambers. Labeled streptavidin fed from an antibody input to sample exhausts binds to the immunostacks. It is to be understood that the operation building the immunostack will be varied as may be required by the scheme of the immunoassay employed. Also the manner of labeling is not limited to the one described, as, for example, direct coupling of fluorescence tags to polyclonal antibodies is possible with commercial kits (e.g. Pierce), thereby circumventing the use of labeled streptavidin. Again pressure is provided to a selected one of control ports to again isolate flow from the portions of the flow channels of chip not used for this purpose. Again the approach is not limited to pneumatically actuated valves.

Sixth, a conventional fluorescence readout is performed. Where the detection mechanism is not fluorescent, the detection step and the detector used will be modified accordingly. The detected fluorescence signal quantifies the captured antigens. In a fluorescence readout a microscope and CCD camera employed, or a micro-CCD array without a microscope, a film plate or any other light detection means can be substituted.

The illustrated embodiment of the invention can thus be characterized as a microfluidic assay apparatus comprising a matrix, a plurality of sample/buffer flow channels defined in the matrix, a plurality of antibody/buffer flow channels defined in the matrix and intersecting the plurality of sample/buffer flow channels, a corresponding plurality of selectively controllable, valved capture microchambers, the capture microchamber being defined at each intersection of the plurality of sample/buffer flow channels and the plurality of antibody/buffer flow channels, means for collecting a protein in the plurality of capture microchambers, and means for detecting the plurality of collected proteins in the capture microchambers.

The means for detecting the plurality of collected proteins in the capture microchambers comprises means for quantifying the concentration of the protein, which is collected in the capture microchambers.

The means for detecting the plurality of collected proteins in the capture microchambers comprises means for qualitatively identifying the protein, which is collected in the capture microchambers.

The means for collecting the protein in the plurality of capture microchambers comprises means for simultaneously collecting a plurality of different kinds of proteins in corresponding different capture microchambers.

The plurality of sample/buffer flow channels are arranged and configured to simultaneously receive a plurality of different samples.

The matrix is comprised of a selectively epoxide coated substrate and at least one PDMS layer disposed on the epoxide coated substrate. Each of the selectively controllable, valved capture microchambers are defined in the at least one PDMS layer and comprise at least one push-down or push-up valve to control flow into or out of the capture microchamber.

In the illustrated embodiment the means for simultaneously collecting a plurality of different kinds of proteins in corresponding different capture microchambers comprises a plurality of antigens that are blood analytes, but the approach is not limited to blood analytes only.

The microfluidic assay apparatus of claim 1 where the plurality of antigens are selectively attached to the substrate by means of selectively accessing epoxide coated substrate surfaces.

One or more of the plurality of sample/buffer flow channels are selectively coupled through selective communication with one or more controllable, valved capture microchambers to form a circulation or oscillation path of fixed volume and further comprising a pump included in the path to circulate or oscillate fluid in the path for a predetermined interval to increase collection of the protein in the at least two capture microchambers.

The plurality of capture microchambers are selectively sized to provide a capture surface, which is scaled according to an expected concentration of protein. Smaller capture surfaces are used for lower expected concentrations, to reduce total integrated background and thus improve signal-to-noise. Larger capture surfaces are used for higher expected concentrations, to allow the capture of more protein without surface saturation.

The microfluidic assay apparatus further comprises means for diluting a sample with a predetermined amount of buffer to adjust the sample concentration into an acceptable range of measurement within the microchambers.

The illustrated embodiment of the invention similarly can be characterized as a method of performing a microfluidic assay comprising the steps of selectively flowing selected monoclonal antibodies in a plurality of horizontal flow channels in a microfluidic, optically transparent, biologically inert matrix, selectively bonding selected monoclonal antibodies to binding moieties on a surface in a corresponding microchambers in the microfluidic matrix, flowing a derivatization buffer in the horizontal flow channels to remove unbound excess protein and to passivate any unreacted binding moieties that would otherwise produce background by binding proteins in later flows, flowing a buffer in vertical flow channels to passivate the vertical flow channels, flowing a plurality of samples in vertical flow channels to fill a corresponding pair of vertical flow channels, circulating a fixed volume of the sample in the pair of vertical flow channels to capture protein by the antibodies in corresponding microchambers, the corresponding microchambers being communicated to the pair of flow channels, flowing buffer in the vertical flow channels to flush out the sample volume with any unbound protein, flowing selected polyclonal antibodies in selected horizontal flow chambers to build up an immunostack in the microchambers, flowing buffer in the horizontal flow channels to remove unattached polyclonal antibody, flowing fluorescently labeled tags in the horizontal flow channels to tag the polyclonal antibody, flowing a buffer in the horizontal flow channels to remove excess unattached tags, and measuring fluorescence detection in the microchambers. Here and henceforth, “horizontal” and “vertical” refer to the orientation of the sets of channels as shown on the attached figures, rather than relative orientation with respect to gravity vector in the physical device.

The step of circulating a fixed volume of the sample in the pair of vertical flow channels to capture protein by the antibodies in corresponding microchambers comprises flowing the fixed volume of the sample along a closed path to maximize extraction of the protein from the sample, by exposing the same capture surface to all sections of the volume one or multiple times.

Prior to flowing a plurality of samples in vertical flow channels to fill a corresponding pair of vertical flow channels, the method further comprises selectively diluting selected ones of the samples with a standard buffer to adjust the sample with a predetermined range of concentrations.

Yet another embodiment of the invention is a method for performing a microfluidic assay comprising the steps of selectively flowing a plurality of antibodies in a plurality of flow channels in communication with a plurality of microchambers in a microfluidic matrix, selectively bonding selected antibodies to binding moieties on a surface of the corresponding microchambers in the microfluidic matrix, flowing a derivatization buffer in the flow channels in the microfluidic matrix to remove unbound excess protein and to passivate any unreacted binding moieties in the microchambers that would otherwise produce background by binding proteins in later flows, flowing a plurality of samples in flow channels communicated to the microchambers in the microfluidic matrix to fill a predetermined volume of the microfluidic matrix, which predetermined volume at least includes the microchambers, bonding a corresponding plurality of proteins to the selected antibodies on the surface in the corresponding microchambers in the microfluidic matrix, flowing buffer in the flow channels to flush out the sample volume with any unbound protein from the microchambers, and measuring bound protein in the plurality of microchambers.

In this last embodiment the step of bonding a corresponding plurality of proteins to the selected antibodies on the surface in the corresponding microchambers in the microfluidic matrix comprises circulating a fixed volume of the sample in the flow channels to capture protein by the antibodies in corresponding microchambers.

In this same last embodiment the method further comprises the steps of flowing a buffer in flow channels to passivate the flow channels prior to bonding the corresponding plurality of proteins to the selected antibodies on the surface in the corresponding microchambers in the microfluidic matrix, and flowing fluorescently labeled tags in the flow channels to the plurality of microchambers to tag the sample and flowing a buffer in the flow channels to remove excess unattached tags prior to measuring bound protein in the plurality of microchambers.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a side cross sectional view of a push-down valve used in the microfluidic circuit of the invention.

FIG. 2 is a top plan view of the microfluidic circuit of the illustrated embodiment.

FIG. 3a is a diagram of a top plan view of a full sized microchamber used in the microfluidic circuit of the invention.

FIG. 3b is a diagram of a top plan view of a reduced sized microchamber used in the microfluidic circuit of the invention.

FIG. 4 is a diagram of an immunoassay stack built up at the microchambers in the microfluidic circuit of the invention.

FIG. 5 is a diagram in top plan view of a layout of a circulating flow path or coliseum in the microfluidic circuit of the invention.

FIG. 6 is a microphotograph of the fluorescence image of a microchamber. for a VEGF test of a 0.3 nM sample produced in the microfluidic circuit of the invention.

FIG. 7 is a bar graph of the specificity for five four selected human blood antigens (CRP, VEGF, PSA, ferritin) and a BSA “negative” control sample, using five corresponding microchambers in the microfluidic circuit of the invention.

FIGS. 8a-8d are graphs of the net output signal read from microchambers in the microfluidic circuit of the invention as a function of antigen concentration for PSA, VEGF, CRP and Ferritin in simple solutions (PBS buffer with 0.1% BSA). Twenty samples of known concentrations were tested in two experiments to produce calibration curves for four antigens: PSA, prostate-specific antigen; VEGF, vascular endothelial growth factor; and CRP, C-reactive protein. These blood analytes are related to inflammation, prostate cancer, long-term iron buildup, and cancer, respectively. The system demonstrated sensitivity at the clinically relevant abundances (with a signal-to-noise ratio >8 at as low as 10 pM) while using only 100 nL per sample for all tests and only 300 nL of antibody per test for all samples.

FIGS. 9a-9d are graphs of calibrations showing the net signal output vs a reference concentration as measured by the illustrated embodiment in human plasma for VEGF, PSA, Ferritin and Thyroglobulin respectively.

FIGS. 10a-10d are graphs of calibrations showing the net signal output vs a reference concentration as measured by the illustrated embodiment in human serum for VEGF, PSA, Ferritin and CRP respectively.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

The illustrated embodiment of the invention is a high-throughput multi-antigen high-specificity high-sensitivity reproducible polydimethylsiloxane (PDMS) microfluidic system 10 for quantifying four representative blood analytes 12 at the clinically relevant levels. It is expressly to be understood that the invention could be realized in different systems for quantifying or identifying different numbers of different analytes and in different types of biological samples other than blood, such as urine, spinal fluid, vaginal secretions, perspiration, saliva, synovial fluid, cerebral fluid, ocular fluid, biopsy samples, and many other tissue sample types where the nanoliter sized sample of the invention make testing possible and practical for the first time. The illustrated embodiment is set forth here only for illustration and concreteness of example.

An active microfluidic matrix 14 utilizes arrays of integrated micromechanical or microhydraulically actuated valves 16 to direct pressure-driven flow and multiplex analyte samples 12 with immunoassay reagents 18. Enzyme-linked immunosorbent assay (ELISA)-like fluorescence immunostacks 20 are formed in the microchambers 22 at the intersections of sample channels 24 and reagent channels 26. The fluorescence signals from these microchambers 22 quantify and identify the captured antigens 28. However, the detection mechanism and the corresponding detector to read the assay may assume many different equivalent forms, such as the use of direct fluorophores instead of fluorophores tags, fluorophores by proxy, chemiluminescence, quantum dots, radioactive tags and the like. Any means by which a radiative quantification can be obtained can be substituted.

The 100-chamber system 10 of the illustrated embodiment conducts five tests for each of ten samples 12 with two replicates per sample-test combination. The number of samples can be significantly increased by extending the size of the matrix 14 according to the teachings of the invention by exploiting the known capabilities of PDMS microfluidic technology. In the illustrated embodiment we chose blood analytes 12 for initial validation of the system 10, because blood tests represent one of the most common examples of routine use of immunoassays. However, it is to expressly understood that any kind of analyte, biological or otherwise, could be subjected to the system 10 and methodology of the invention.

The current standard clinical macrofluidic technology is typically based on an enzyme-linked immunosorbent assay (ELISA) and in practice requires 0.5 to 2 mL of sample per test per patient. By contrast with the high sample volumes required by conventional ELISA, the system described here uses only 100 nL of sample for ten tests, while simultaneously measuring CRP, PSA, ferritin, and VEGF within the clinically significant range. The system also uses only 300 nL of antibodies (as low as 0.8 ng) per assay to measure all ten samples. Therefore, the microfluidic miniaturization of immunoassays described here paves the way to efficient and portable hand-held devices to be used by the attending medical staff in the field or in the clinic at the time of sampling.

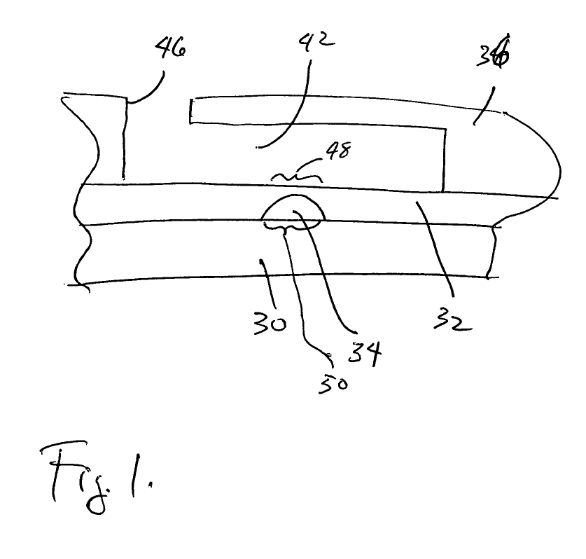

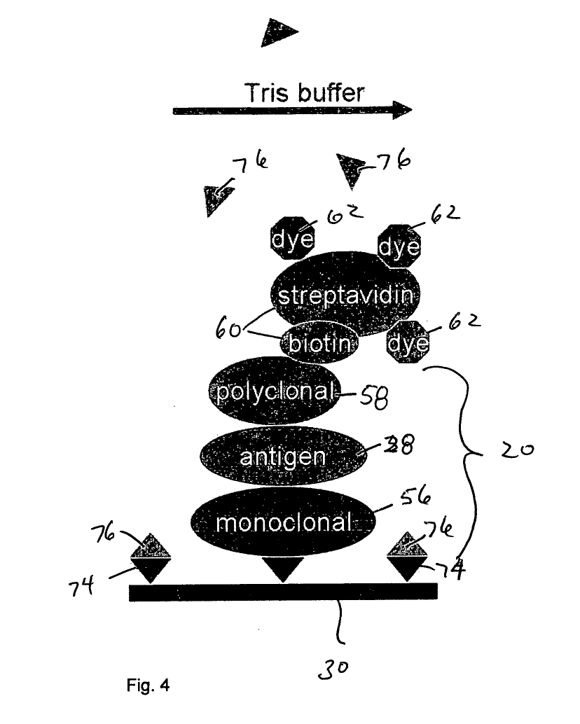

FIG. 1 is a diagram showing a side cross sectional view of a portion of matrix 14, which in the illustrated embodiment is built up on a glass substrate 30, which is selectively coated with epoxide 74 or have other adhesion promotion substances coated on its upper surface to promote molecular attachment of antibodies 56 to substrate 30 as discussed in connection with FIG. 4. A thin molded layer 32 of PDMS is then disposed, attached, molded onto a thicker control layer 36. Layer 32 is the flow layer in which flow channels 24 are defined or molded as described below. Control layer 36 and flow layer 32 are then bound to epoxide-coated substrate 30. PDMS is preferred because it is optically transparent in the visible spectrum, is biologically inert, and can be easily molded or formed using conventional soft lithography. Layer 32 may typically be in the range of 26 to 49 μm thickness.

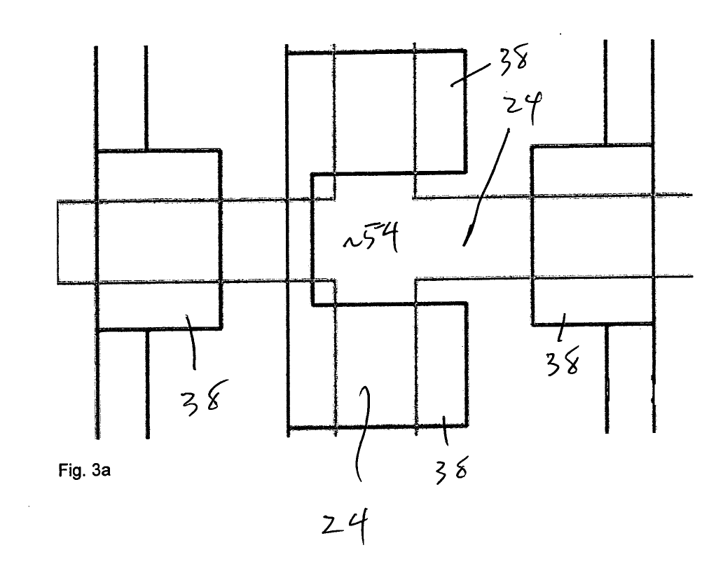

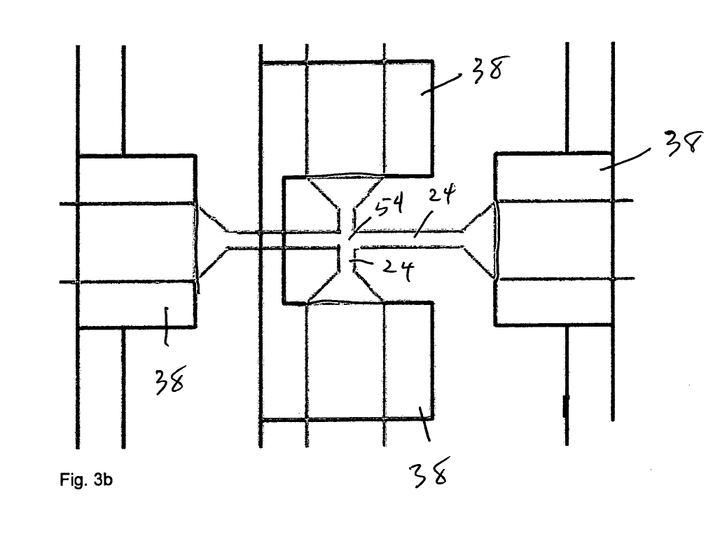

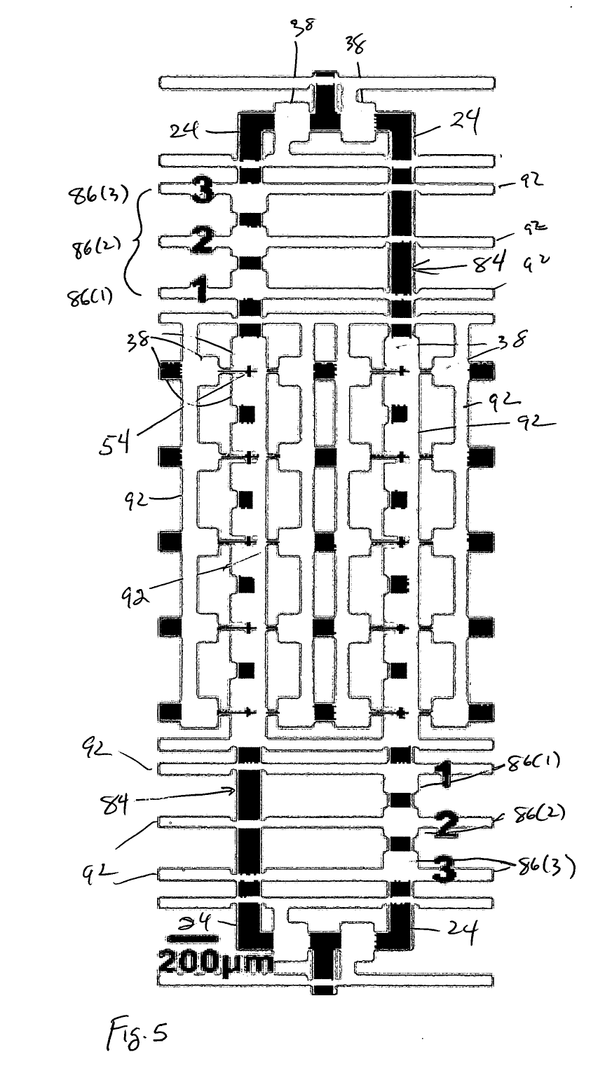

A plurality of deformable valve chambers 34 are defined in layer 32, which can be employed as hydraulically actuated push-down or push-up valves 38, depending on the structure chosen, or combined to function as pumps 40 as described in connection with FIG. 5. FIG. 1 illustrates a single push down valve 38. Valve chamber or valve microchamber 34 will typically be defined in a flow channel 24, best shown in the views of FIGS. 3a and 3b. A thicker control layer 36 of PDMS is disposed on layer 32 into which is defined a pressure chamber 42 communicated by a control channel 92 to a control port 46. Hydraulic fluid is pumped by an exterior pump or pumps, not shown, into pressure chamber 42 thereby forcing down the thinner portion 48 of layer 32 overlying the pushdown valve 38 of which valve chamber 34 is part. By this means, any flow channel 24 in which valve 38 is defined can be selectively shut or closed off by selective pressurization of pressure chamber 42, thereby pushing down portion 48 of layer 32 against portion 50 of substrate 30.

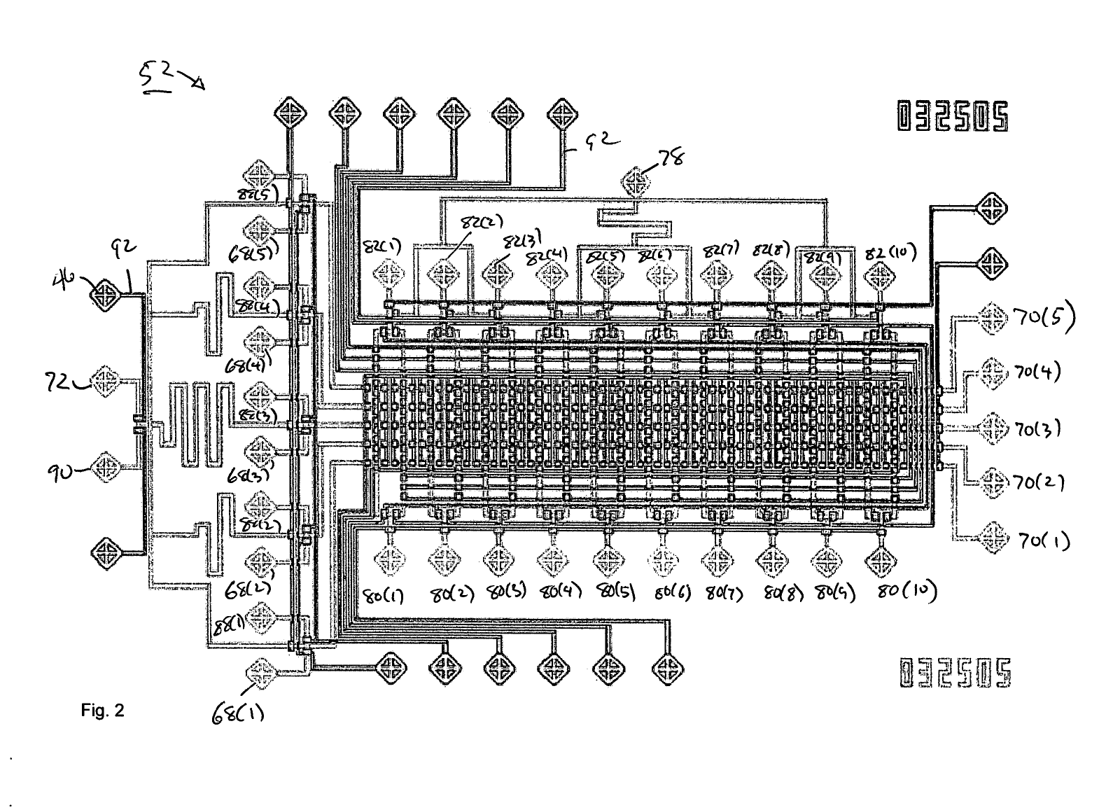

In the illustrated embodiment, a microfluidic circuit 52 in matrix 14 is shown in top plan view of FIG. 2 and is formed by the method described below. Selected positions 54 on the treated upper surface of substrate 30 are provided with selected reagents according to the assay which is to be performed at that site. Positions 55 will underlie intersections of overlying flow channels 24 and comprise the floor of capture microchambers 54. FIGS. 3a and 3b illustrate a top plan view two types of orthogonal intersections of two channels 24 in circuit 52. The site of the intersection defines the selected position 55 and the corresponding valved capture microchamber 54, where a reagent will be selectively provided on substrate 30 by means of selective coating of substrate 30 by an epoxide or other molecular adhesion agent at positions 55 and where a test will be performed.

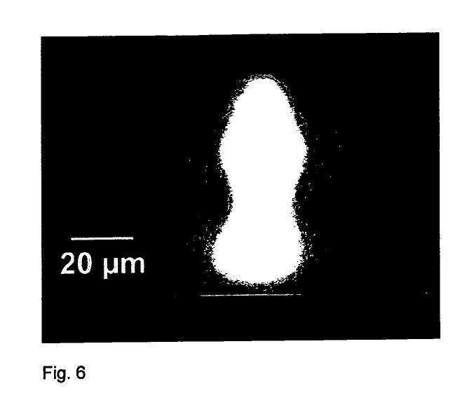

Before considering the fabrication of circuit 52 of FIG. 2 consider first the basic scheme by which immunoassays are made in circuit 52. In sandwich immunoassays, a monoclonal antibody 56 as diagrammed in FIG. 4, specific to the target analyte (antigen 28), is bound to a position or surface 55 in capture microchamber 54 by molecular bonding to an epoxide coating selective provided on surface 55. Different antigens 28 are attached by selectively flowing a solution carrying the chosen antigen 28 through the corresponding horizontal flow channel 24 from one of the antigen ports 88(1)-88(5) described in connection with FIG. 2. Next, the sample 12 is put in contact with surface 54 by selectively flowing a solution carrying the sample 12 through the corresponding vertical flow channel 24 from one of the sample ports 82(1)-82(10) described in connection with FIG. 2, whereby the antibody 56 captures the contained antigen 28. Then, a labeled polyclonal antibody 58 is provided by selectively flowing a solution carrying the chosen polyclonal antibody 58 through the corresponding horizontal flow channel 24 from one of the antigen ports 88(1)-88(5) described in connection with FIG. 2, which attaches to the antigen 28 to complete the immunostack 20. The label (e.g., a linked enzyme 60 creating fluorescent product or a fluorophore 62 bound to the polyclonal antibody 58) generates a light signal using conventional fluoroscopic detection techniques that is compared with a standard to quantify the captured antigen 28. FIG. 6 is a microphotograph which shows a typical fluorescence image from microchamber 54 formed by 20 μm-wide channels for a VEGF test of 0.3 nM sample.

Consider now a listing of reagents and materials used in chip fabrication in the illustrated embodiment. The materials used in chip fabrication included Hexamethyldisilazane (HMDS) adhesion promoter applied to substrate 30 obtained from ShinEtsuMicroSi (Phoenix, Ariz., USA). The photoresist used in chip fabrication (Shipley SJR 5740) was obtained from MicroChem (Newton, Mass., USA). Tetramethyl-chlorosilane (TMCS) was obtained from Sigma (St. Louis, Mo., USA). PDMS Sylgard 184 was obtained from Dow Corning (Midland, Mich., USA). Arraylt® SuperEpoxide SME slides for substrate 30 were obtained from TeleChem International (Sunnyvale, Calif., USA). It must of course be understood that this list of materials is not a limitation on the kinds of materials that can be used to fabricate circuits 52.

Turn now to the antibodies and antigens relevant to the illustrated embodiment. PSA antigen, monoclonal PSA antibody, ferritin antigen, monoclonal ferritin antibody, and monoclonal CRP antibody were procured from Fitzgerald Industries (Concord, Mass., USA); VEGF antigen and antibodies and biotinylated CRP antibody from R&D Systems (Minneapolis, Minn., USA); PSA biotinylated antibody from Lab Vision (Fremont, Calif., USA); ferritin biotinylated antibody from U.S. Biological (Swampscott, Mass., USA); and CRP antigen from EMD Biosciences (Calbiochem®; San Diego, Calif., USA). Again, it must be explicitly understood that this list of antibodies and antigens is not a limitation on the kinds of biological compounds that can be used or tested in circuits 52.

Finally, consider the fluorescent probes and buffers used in the illustrated embodiment. Streptavidin Alexa Fluor® 555 was supplied by Invitrogen (Molecular Probes™; Carlsbad, Calif., USA). Lyophilized commercial antigens and antibodies were reconstituted in phosphate-buffered saline (PBS) 1× buffer from Irvine Scientific (Santa Ana, Calif., USA), pH 7.5. Bovine serum albumin (BSA) was added to the same to produce the PBS 0.1% BSA buffer. The passivation buffer was 10 mM Tris, 10 mM NaCl, pH 8.0, made from powdered Tris and NaCl (both from Sigma). It must be explicitly understood that this list of fluorescent probes and buffers is not a limitation on the kinds of probes and buffers that can be used or tested in circuits 52.

Consider now the general method of making a mold for a circuit 52 such as that shown in FIG. 2. PDMS microfluidic chips 52 with integrated micro-mechanical valves 38 were built using conventional soft lithography with the following modifications. Silicon wafers used as a negative mold were exposed to HMDS vapor for 3 min. The silicon wafers were coated with Photoresist SPR 220-7 by spinning at 2000 rpm for 60 s on a WS-400A-GNPP/LITE spincoater (Laurell Technologies, North Wales, Pa., USA). The silicon wafers were baked at 105° C. for 90 s on a hotplate. UV exposure through black-and-white transparency masks was performed for 1.75 min on a Karl Suss MJB3 mask aligner (Karl Suss America, Waterbury, Vt., USA). The molds were then developed for 2 min in 100% MicroChem 319 developer (MicroChem). Flow layer molds were baked at 140° C. for 15 min on a hotplate to melt and round the flow channels 24. Molds were characterized on an Alpha-Step 500 (KLA-Tencor, Mountain View, Calif., USA). Channel height was between 9 and 10 μm. The control channel 92 profile was rectangular, while the flow channel 24 profile was parabolic. Except for the height measurements, the mold fabrication was conducted in a Class 10,000 clean room.

The molds were exposed to TMCS vapor for 3 min. PDMS in 5:1 and 20:1 ratios were mixed and degassed using an HM-501 hybrid mixer and cups from Keyence (Long Beach, Calif., USA). Thirty-five grams of the 5:1 were poured onto the control mold used to make the control layer 36 in a plastic Petri dish wrapped with aluminum foil. Five grams of the 20:1 were spun over the flow mold at 1500 rpm for 60 s using a P6700 spincoater from Specialty Coating Systems (Indianapolis, Ind., USA). Both were baked in an 80° C. oven for 30 min. The control layer 36 was taken off its mold and cut into respective chips pieces or portions. Control line ports 46 were punched using a 20-gauge Intramedic™ Luer-Stub adapter (BD Biosciences, Franklin Lakes, N.J., USA). Control layer 36 pieces were washed with ethanol, blown dry with filtered air or nitrogen, and aligned on top of the flow layer 32 under a stereoscope. The result was baked in an 80° C. oven for 1 h. Chip pieces were then cut out and peeled off the flow layer 32 mold. Flow line ports 68(i), 70(i), 72, 78, 80(i), 82(i), 88(i), and 90 shown in FIG. 2 were punched with the 20-gauge Luer-stub adapter. Chip pieces were then washed in ethanol and blown dry before binding to the epoxide glass slides 30. The assembled chips 52 of FIG. 1 underwent a final bake overnight in an 80° C. oven.

To bench test the assembled chip 52 an inverted Olympus IX50 microscope (Olympus America, Melville, N.Y., USA) was equipped with a mercury lamp (HBO® 103 W/2; Osram, Munich, Germany), an Olympus Plan 10× objective [numerical aperture (NA) 0.25], a long-distance Olympus SLCPlanFI 40× objective (NA 0.55), a cooled charge-coupled device (CCD) camera (Model SBIG ST-71; Santa Barbara Instrument Group, Santa Barbara, Calif., USA), and a fluorescence filter set (excitation: D540/25, dichroic 565 DCLP; emission: D605/55) from Chroma Technology (Brattleboro, Vt., USA). We then plugged 23-gauge steel tubes from New England Small Tube (Litchfield, N.H., USA) into the chip's control channel ports 46 described below. Their other ends were connected through Tygon® tubing (Cole-Parmer, Vernon Hills, Ill., USA) to Lee-valve arrays (Fluidigm, San Francisco, Calif., USA) operated by LabView software on a personal computer. The same types of steel tubes and Tygon plumbing were used to supply reagents to the chip's flow channel ports 66 described below. It is to be understood that the illustrated embodiment is a bench prototype and that the elements of a control system for providing pressurized control fluid, antigens, buffers, samples and the like will be modified from that disclosed to be optimized and miniaturized in the commercial production system according to conventional engineering design principles.

The immunoassay and the fabrication of chip 52 having been described, it is possible now to consider the implementation of chip 52 in matrix 14 according to the invention as shown in the example of FIG. 2. The microfluidic immunoassays chip 52 of FIG. 2 includes in the illustrated embodiment a 100-chamber, 22×35 mm PDMS chip 52 bound to an epoxide slide 30 to simultaneously perform five tests each of ten samples, with two chambers 54 per sample-test combination. Control channels 92 are shown in dark outline and convey hydraulic pressure to open and close microvalves 38 that direct pressure-driven feeds of reagents along flow channels 24 according to the valve action as described in connection with in FIG. 1. Each intersection of flow channels 24 in the central test matrix 14 forms a microchamber 54 where an immunostack 20 is constructed as described above. The flow channels are described below as comprised of horizontal and vertical flow channels 24 and are shown as such in FIGS. 2, 3a and 3b. However, it is to be understood that orientation with respect to any defined direction is immaterial, that the intersections of flow channels 24 need not be orthogonal, but may be skewed, and in fact microchambers 54 and channels 24 need not be rasterized in a matrix array, but topologically organized in any arrangement consistent with the teachings of the invention. Nevertheless, it is the preferred embodiment to have channel and microchamber layout which is both rasterized and orthogonal for ease of manufacture and scaling the matrix size or number up or down as desired.

Turn now to the layout of circuit or chip 52 of the illustrated embodiment as shown in plan view in FIG. 2 and consider the steps summarized above taken in a measurement or assay in more detail. In a typical measurement, monoclonal antibodies 56 flow in horizontal flow channels 24 from derivatization inputs 68(1)-68(5) to derivatization exhausts 70(1)-70(5) in FIG. 2. The antibodies 56 covalently bond to the epoxide floor 54 of the microchannels 24, producing the first layer of the immunostack 20 in FIG. 4. Tris buffer 76 flowing from derivatization buffer input 72 flows in horizontal flow channels 24 to derivatization exhausts 70(1)-70(5) to remove unbound excess protein and passivates any unreacted epoxide moieties 74 that would otherwise produce background by binding protein in later feeds. Next, Tris buffer 76 flows from samples buffer input 78 in vertical flow channels 24 to samples exhausts 80(1)-80(10) to passivate the rest of the microchannels 24.

As samples 12 flow in parallel in vertical flow channels 24 from sample inputs 82(1)-82(10) to sample exhausts 80(1)-80(10), each sample 12 fills a corresponding pair of microchannels 24. When the appropriate valves 38 are closed, each such pair of microchannels 24 forms a closed path, called here a coliseum 84, that traps 10 nL of the respective sample 12 as shown in better view in FIG. 5. Then, an array of peristaltic micropumps 86(i), which are three cyclically driven valves as shown in FIG. 1, drive each trapped volume around its coliseum 84, with a lap time of 20 s. A cyclical application of pressure is provided to micropumps 86(i) through control flow channels 92 from three corresponding control ports 46 provided from an exterior pump and controller (not shown). Within each coliseum 84, each antigen 28 is captured in its respective microchamber 54, as determined by the first layer of the immunostack 20. The same sample 12 is allowed to run multiple laps (typically 10) to maximize extraction of the antigen 28 from the sample 12.

FIG. 5 is a functional diagram of an individual coliseum 84. Here control channels 92 and valves 38 are drawn in dark outline and flow channels 24 in lighter outline. Comb like valve arrays 86(1)-86(3) enclose a pair of immunoassay chambers 54 for each of five tests. Valve arrays 86(1)-86(3) pump the sample 12 in a circle along the coliseum 84 [e.g., clockwise for actuation order (86(1), 86(2), 86(3)] with a lap time of 20 s. Again pressure is provided to a selected one of control ports 46 to again isolate flow from the portions of the flow channels 24 of chip 52 not used for this purpose.

After harvesting, buffer 76 from samples buffer port 78 flow in vertical flow channels to sample exhaust ports 80(1)-80 (10) to flush out the sample volume. Parallel feeds of biotinylated antibodies 58 from antibody inputs 88(1)-88(5) flow in horizontal flow channels 24 to derivatization exhausts 70(1)-70(5) respectively to build up the third layers of the immunostacks 20 in each microchamber 54. Buffer 76 from derivatization buffer input 72 flow in horizontal flow channels 24 to derivatization exhausts 70(1)-70(5) to remove unattached antibody 58. Fluorescently labeled streptavidin 60 in PBS buffer flows from streptavidin input 90 in horizontal flow channels 24 to derivatization exhausts 70(1)-70(5). Then, buffer from derivatization buffer input 72 flows in horizontal flow channels 24 to derivatization exhausts 70(1)-70(5) to remove unattached excess. All valves 38 are then closed, and fluorescence detection is conducted at each microchamber 54 using an inverted optical microscope and an inexpensive, cooled CCD camera or other detection means, which produces an image as shown in FIG. 6 of each chamber 54.

Chip 52 now having been described and its fabrication disclosed, consider the performance of the illustrated embodiment with respect to blood protein assays. Blood proteins were chosen to validate the system because blood tests are one of the most common and clinically important applications of immunoassays. In particular, CRP, PSA, ferritin, and VEGF were selected due to their significance in medical diagnostics, the wide concentration range spanned by their clinically normal levels, and the commercial availability of well-validated antigens and antibodies.

To test the specificity of the system, we processed one load of 10 nL for each of four samples, each containing 20 nM of one of the antigens in PBS 0.1% BSA, in a chip with 100 μm-wide channels (approximately 50,000 μm² per microchamber 54). Because every test lane intersects every coliseum 84 in a pair of microchambers 54, the fluorescence signals of each such pair 54 were added to produce the signal for the respective sample-test combination. After normalizing for area, we divided each signal by the fluorescent background of the particular test as measured in regions unexposed to antigen.

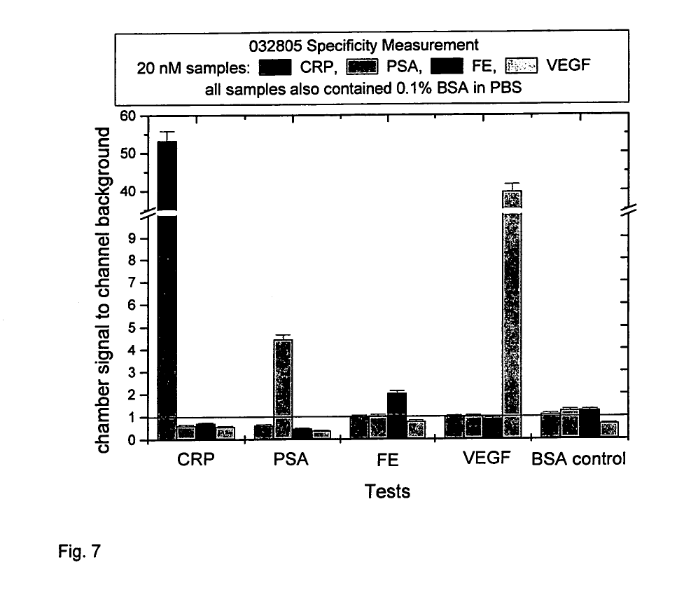

The results are graphed in FIG. 7. Samples 12 each containing 20 nM of a single antigen, CRP, PSA, ferritin, and VEGF, were fed in parallel into the test matrix 14. Each sample 12 produced significant signal above background only in the test corresponding to the antigen contained in the sample 12. The BSA control produced signal at the background level. The results showed the specificity of measurement and the lack of crosstalk between tests. Every sample 12 produced significant signal above background only in the test chambers 54 corresponding to the antigen 28 it contained. In addition, the presence of the antigens 28 does not increase the background in the control case, where PBS 0.1% BSA replaced the antibody feeds. These results demonstrate the specificity of the system.

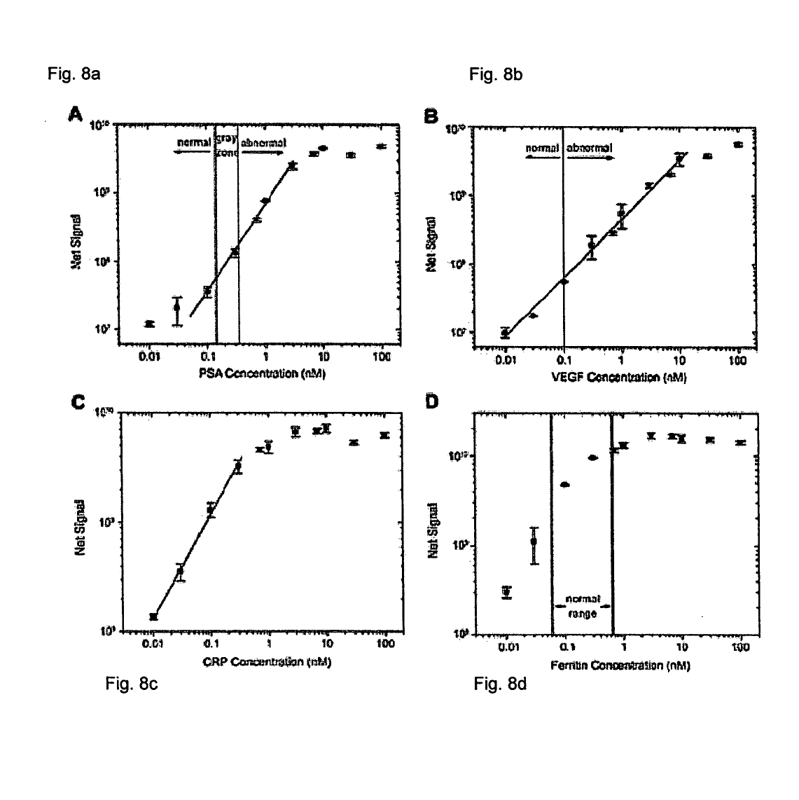

To test the sensitivity of the system, we ran 10 samples against the same four tests but in devices with 20 μm-wide channels 24 at the intersections (approximately 2000 μm² per microchamber 54). One sample was a control containing no antigen 28. Each of the other nine samples contained all antigens at the same concentration, which was varied between 30 pM and 10 nM from sample to sample, all in PBS 0.1% BSA. We processed 100 nL per sample (10 loads of 10 nL). The signal for each sample-test was extracted from fluorescence images of the chambers by subtracting the local background for each image and adding the two such results per sample-test combination. Then, for each test, the signal of the control sample was subtracted from the signals of the other nine samples to produce the final results for each test. To establish reproducibility, the same experiment was repeated in another chip with a new dilution of reagents. Also, the concentration range was expanded (10 pM to 100 nM). Data analysis was conducted as described above. The results were combined in a single plot per test as graphed in FIGS. 8a-8d, including the clinically relevant levels (www.labtestsonline.org). The data demonstrate the reproducibility of results in the system.

The net signal for the lowest concentration (10 pM) for each test was divided by the uncertainty of the respective control signal to produce a measure of the observed signal to noise. The results were 164 (CRP), 38 (PSA), 11 (ferritin), and 8 (VEGF).

The PSA test shows a linear calibration between 100 pM and 30 nM in FIG. 8a. This dynamic range includes the “gray zone” at 4.0-10.0 ng/mL (133-332 pM). The higher the concentration above the “gray zone,” the stronger the indication for prostate cancer. Similarly, VEGF has a linear calibration between 10 pM and 10 nM in FIG. 8b. This range includes the important cutoff at 25 ng/mL (0.1 nM), exceeding which is an indication for cancer.

The CRP test shows that the system is linear between 10 and 300 pM, after which the signal saturates in FIG. 8c. In this case, the sensitivity is excessive since the clinically abnormal levels are above 1.2 mg/dL (110 nM), indicating acute infection. Similarly, the ferritin detection in FIG. 8d is sensitive within and below the normal range of 30-300 ng/mL (60-630 pM) but saturates above it, where long-term iron buildup is indicated.

The observed saturation for CRP and ferritin can be avoided in a number of straightforward ways, producing chips 52 customized to a particular set of tests. In such chips 52, the scarce-agent tests would retain the smallest channels 24 for maximal sensitivity as shown in FIG. 3b, while the abundant-agent tests would have wider channels to increase capture area and thus raise the saturation point as shown in FIG. 3a. Tests for ultra-abundant agents (e.g., ceruloplasmin, normally at 21-50 mg/dL) would be organized in another section of the chip 52 and would be preceded by a dilution stage to reduce the concentration into the measurable range. Because the dilution factor would be predetermined by the device geometry, straightforward multiplication would yield the correct final result.

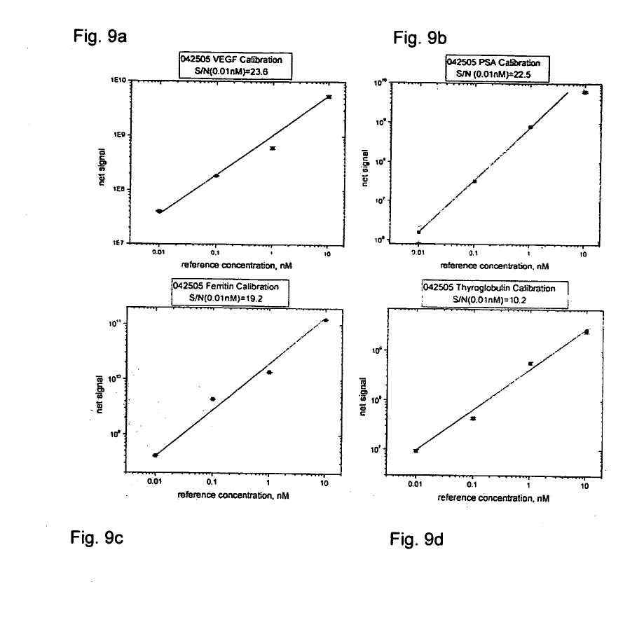

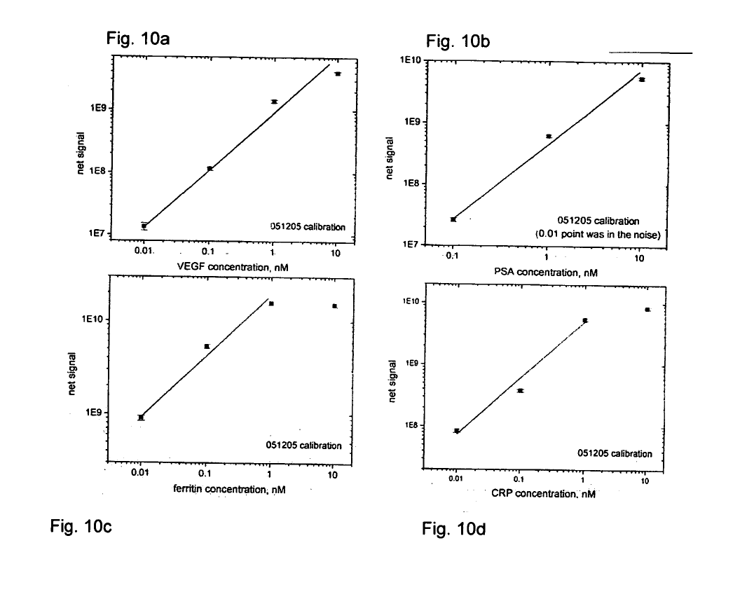

FIGS. 9a-9b and 10a-10b show the human plasma and serum data respectively which illustrates the system of the invention functions correctly with real human serum/plasma samples and moreover, reproduces the same calibration curves as with simple solutions. In this case, the human samples were spiked with commercially available pure antigens.

The parsimony of the system 10 is important in any immunoassay application where sample 12 is costly or scarce. In blood tests, the current practical requirement is 0.5-2 mL per sample per test, necessitating drawing blood from the vein and making common blood tests difficult for pediatric patients. In contrast, the system presented here uses 100 nL of each sample for all tests, thus enabling the development of portable apparatuses conducting common blood tests by a finger prick.

Simultaneously, the system uses 300 nL (as low as 0.8 ng) of antibody per sample-test combination. In contrast, the state-of-the-art Elecsys® PSA kit from Roche Applied Science (Indianapolis, Ind., USA) uses 200 ng per sample-test, or 250 times more. The savings have direct consequences in modern healthcare and biomedical research.

The produced calibration curves could be used as the established dependences, which allow internal recalibrations to be constructed within each measurement by running just a few reference samples per device. This technique would eliminate systematic sources of variation, such as quality and condition of reagents, intensity of the illumination source, and differences in storage and handling. Simultaneously, the results would be extended to more complex media, such as human serum, plasma, spinal fluid, and biopsy samples. Finally, the test matrix 14 could be expanded to 50×50 in commercial products.

The illustrated embodiment demonstrates the reduction of immunoassays to a microfluidic high-throughput multi-antigen format. The developed system 10 is an important step toward derivative immunoassay applications in scientific research and point-of-care testing in medicine.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

Thus, it can now be appreciated that the invention can be reproduced in a portable, field usable unit which can provide high-throughput, multi-antigen tests at low cost. Blood samples in the volume of pin pricks can be utilized without the need for a qualified phlebotomist. A multiple number of patients may be tested using the same matrix with one sample input being taken from each patient. Testing can be done at the surgery site on a continual basis without need to delay or wait for conventional remote lab testing. As the number of proteins discovered increases or their significance to physiological function is discovered, the apparatus of the invention can accommodate significant expansion in the number of analytes testing, which increased numbers would overwhelm and overrun conventional testing apparatus and procedures both in terms of cost, time and feasibility. The specificity and calibration of the methodology and apparatus of the invention easily meets and exceeds current clinical standards and even promises to raise those standards in many cases. At the same time, the invention is noninvasive and utilizes conventional immunoassays, thereby avoiding lengthy or complex FDA approvals. The use of the apparatus is simple and inexpensive enough to conveniently allow for patient self-monitoring in patients suffering from diabetes, cancer, cardiovascular diseases or those seeking hormonal or metabolic health, performance or fitness. Finally, the invention lends itself to system integration so that it can be practically and readily rendered a plurality of packages and applications.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. A microfluidic assay apparatus comprising:
a matrix;
a plurality of sample/buffer flow channels defined in the matrix;
a plurality of antibody/buffer flow channels defined in the matrix and intersecting the plurality of sample/buffer flow channels;
a corresponding plurality of selectively controllable, valved capture microchambers, the capture microchamber being defined at each intersection of the plurality of sample/buffer flow channels and the plurality of antibody/buffer flow channels;
means for collecting a protein in the plurality of capture microchambers; and
means for detecting the plurality of collected proteins in the capture microchambers.

2. The microfluidic assay apparatus of claim 1 where the means for detecting the plurality of collected proteins in the capture microchambers comprises means for quantifying the concentration of the protein, which is collected in the capture microchambers.

3. The microfluidic assay apparatus of claim 1 where the means for detecting the plurality of collected proteins in the capture microchambers comprises means for qualitatively identifying the protein, which is collected in the capture microchambers.

4. The microfluidic assay apparatus of claim 1 where means for collecting the protein in the plurality of capture microchambers comprises means for simultaneously collecting a plurality of different kinds of proteins in corresponding different capture microchambers.

5. The microfluidic assay apparatus of claim 1 where the plurality of sample/buffer flow channels are arranged and configured to simultaneously receive a plurality of different samples.

6. The microfluidic assay apparatus of claim 1 where the matrix is comprised of a selectively epoxide coated substrate and at least one PDMS layer disposed on the epoxide coated substrate.

7. The microfluidic assay apparatus of claim 6 where each of the selectively controllable, valved capture microchambers are defined in the at least one PDMS layer and comprise at least one push-down or pull-up valve to control flow into or out of the capture microchamber.

8. The microfluidic assay apparatus of claim 1 where the means for simultaneously collecting a plurality of different kinds of proteins in corresponding different capture microchambers comprises a plurality of antigens.

9. The microfluidic assay apparatus of claim 1 where the plurality of antibodies are selectively attached to the substrate by means of selectively epoxide coated substrate surfaces.

10. The microfluidic assay apparatus of claim 1 where ones of the plurality of sample/buffer flow channels are selectively coupled through selective communication of at least two controllable, valved capture microchambers to form a circulation path of fixed volume and further comprising a pump included in the circulation path to circulate fluid in the path for a predetermined interval to increase collection of the protein in the at least two capture microchambers.

11. The microfluidic assay apparatus of claim 1 where one of the plurality of sample/buffer flow channels is selectively communicated by selective valve actuation to a selected capture microchamber and portion of the communicated sample/buffer flow channel to form a path of fixed volume and further comprising a pump included in the path to flow fluid in the path for a predetermined interval to increase collection of the protein in the at the selected capture microchamber.

12. The microfluidic assay apparatus of claim 1 where the plurality of capture microchambers are selectively sized to provide a capture surface, which is scaled according to an expected concentration of protein.

13. The microfluidic assay apparatus of claim 12 where the capture surface is smaller, the lower is the expected concentration of protein.

14. The microfluidic assay apparatus of claim 1 further comprising means for diluting a sample with a predetermined amount of buffer to adjust the sample concentration into an acceptable range of measurement within the microchambers.

15. A method of performing a microfluidic assay comprising:
selectively flowing selected monoclonal antibodies in a plurality of horizontal flow channels in a microfluidic, optical transparent, biologically inert matrix;
selectively bonding selected monoclonal antibodies to binding moieties on a surface in a corresponding microchambers in the microfluidic matrix;
flowing a derivatization buffer in the horizontal flow channels to remove unbound excess protein and to passivate any unreacted binding moieties that would otherwise produce background by binding proteins in later flows;
flowing a buffer in vertical flow channels to passivate the vertical flow channels;
flowing a plurality of samples in vertical flow channels to fill a corresponding pair of vertical flow channels;
circulating a fixed volume of the sample in the pair of vertical flow channels to capture protein by the antibodies in corresponding microchambers, the corresponding microchambers being communicated to the pair of flow channels;
flowing buffer in the vertical flow channels to flush out the sample volume with any unbound protein;
flowing selected polyclonal antibodies in selected horizontal flow chambers to build up an immunostack in the microchambers;
flowing buffer in the horizontal flow channels to remove unattached polyclonal antibody;
flowing fluorescently labeled tags in the horizontal flow channels to tag the polyclonal antibody;
flowing a buffer in the horizontal flow channels to remove excess unattached tags; and
measuring fluorescence detection in the microchambers.

16. The method of claim 15 where circulating a fixed volume of the sample in the pair of vertical flow channels to capture protein by the antibodies in corresponding microchambers comprises flowing the fixed volume of the sample a closed path to maximize extraction of the protein from the sample.

17. The method of claim 15 where prior to flowing a plurality of samples in vertical flow channels to fill a corresponding pair of vertical flow channels the method further comprises selectively diluting selected ones of the samples with a standard buffer to adjust the sample with a predetermined range of concentrations.

18. A method of performing a microfluidic assay comprising:
selectively flowing a plurality of antibodies in a plurality of flow channels in communication with a plurality of microchambers in a microfluidic matrix;
selectively bonding selected antibodies to binding moieties on a surface of the corresponding microchambers in the microfluidic matrix;
flowing a derivatization buffer in the flow channels in the microfluidic matrix to remove unbound excess protein and to passivate any unreacted binding moieties in the microchambers that would otherwise produce background by binding proteins in later flows;
flowing a plurality of samples in flow channels communicated to the microchambers in the microfluidic matrix to fill a predetermined volume of the microfluidic matrix, which predetermined volume at least includes the microchambers;
bonding a corresponding plurality of proteins to the selected antibodies on the surface in the corresponding microchambers in the microfluidic matrix;
flowing buffer in the flow channels to flush out the sample volume with any unbound protein from the microchambers; and
measuring bound protein in the plurality of microchambers.

19. The method of claim 18 where bonding a corresponding plurality of proteins to the selected antibodies on the surface in the corresponding microchambers in the microfluidic matrix comprises circulating a fixed volume of the sample in the flow channels to capture protein by the antibodies in corresponding microchambers.

20. The method of claim 18 further comprising:
flowing a buffer in flow channels to passivate the flow channels prior to bonding the corresponding plurality of proteins to the selected antibodies on the surface in the corresponding microchambers in the microfluidic matrix; and
flowing fluorescently labeled tags in the flow channels to the plurality of microchambers to tag the sample and flowing a buffer in the flow channels to remove excess unattached tags prior to measuring bound protein in the plurality of microchambers.
